(19) United States
(12) Patent Application Publication
(10) Pub. No.: US 2009/0099443 A1
(43) Pub. Date: Apr. 16, 2009

(54) MAGNETIC RESONANCE DEVICE AND METHOD

(75) Inventors: Jurgen Erwin Rahmer, Hamburg (DE); Peter Boernert, Hamburg (DE)

Correspondence Address: PHILIPS INTELLECTUAL PROPERTY & STANDARDS, 595 MINER ROAD, CLEVELAND, OH, 44143, US

(73) Assignee: Koninklijke Philips Electronics N.V., Eindhoven (NL)

(21) Appl. No.: 12/297,650

(22) PCT Filed: Apr. 17, 2007
(86) PCT No.: PCT/IB07/51358

§ 371 Date: Oct. 20, 2008

(30) Foreign Application Priority Data
Apr. 21, 2006 (EP) 06112887.2

(51) Int. Cl. A61B 5/055

(52) U.S. Cl. 600/410

(57) ABSTRACT

The invention relates to a device for MR imaging of a body (7) placed in an examination volume. The device (1) comprises means (2) for establishing a substantially homogeneous main magnetic field in the examination volume, means (3, 4, 5) for generating switched magnetic field gradients superimposed upon the main magnetic field, means (6) for radiating RF pulses towards the body (7), control means (12) for controlling the generation of the magnetic field gradients and the RF pulses, means (10) for receiving and sampling MR signals, and reconstruction means (14) for forming MR images from the signal samples. In accordance with the invention, the device (1) is arranged to a) generate a series of MR signals by subjecting at least a portion of the body (7) to an MR imaging sequence of at least one RF pulse and switched magnetic field gradients, the switched magnetic field gradients being selected such that a substantially spherical volume in k-space is sampled along a plurality of radial directions having a non-isotropic angular spacing, the angular density of the radial k-space directions being reduced in the polar regions of the spherical volume, and b) acquire the MR echo signals for reconstructing an MR image therefrom.

The invention relates to a device for magnetic resonance imaging of a body placed in an examination volume.

Furthermore, the invention relates to a method for MR imaging and to a computer program for an MR device.

In magnetic resonance imaging (MRI) pulse sequences consisting of RF pulses and switched magnetic field gradients are applied to an object (a patient) placed in a homogeneous magnetic field within an examination volume of an MR device. In this way, k-space is sampled and magnetic resonance signals are generated, which are scanned by means of RF receiving antennas in order to obtain information from the object and to reconstruct images thereof. Since its initial development, the number of clinically relevant fields of application of MRI has grown enormously. MRI can be applied to almost every part of the body, and it can be used to obtain information about a number of important functions of the human body. The pulse sequence, which is applied during an MRI scan, plays a significant role in the determination of the characteristics of the reconstructed image, such as location and orientation in the object, dimensions, resolution, signal-to-noise ratio, contrast, sensitivity for movements, etcetera. An operator of an MRI device has to choose the appropriate sequence and has to adjust and optimize its parameters for the respective application.

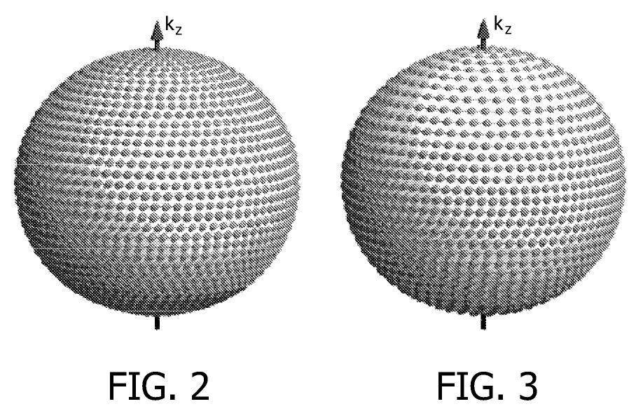

Known three-dimensional (3D) radial sampling schemes allow the acquisition of spherical sampling volumes in k-space with isotropic resolution. Such techniques have been applied to MR cardiac imaging and angiography for its relative insensitivity to motion, but also to ultrashort echo-time imaging (UTE). With UTE imaging, the free-induction decay (FID) is sampled without the necessity of phase encoding. The application of a typical 3D UTE sequence is known, e.g., from a publication by J. Rahmer et al. (J. Rahmer, P. Börnert, C. Schröder, C. Stehning, Proc. Intl. Soc. Mag. Reson. Med., 12 (2004), 2345). This known 3D radial technique samples k-space with isotropic angular density, which can be obtained by arranging radial profiles on a spiral path over the surface of a sphere. Such a conventional 3D radial sampling scheme is illustrated in FIG. 2.

The benefits of known 3D radial sampling schemes, such as good motion properties and isotropic 3D image resolution are counterbalanced by the necessity to acquire a large number of radial profiles to obtain aliasing-free images. This results in long scan durations and large amounts of acquired data. The latter problem is strongly aggravated in multicoil imaging, where the amount of acquired data is proportional to the number of receive coils. One approach to overcome the problems is strong angular undersampling, which, however, leads to an increased level of radial streaking artifacts in the image.

Therefore, it is readily appreciated that there is a need for an improved 3D radial sampling technique. It is consequently an object of the invention to provide an MR device that enables 3D radial sampling of k-space with increased imaging speed and with a tolerable level of image artifacts.

In accordance with the present invention, an MR device for magnetic resonance imaging of a body placed in an examination volume is disclosed, which comprises means for establishing a substantially homogeneous main magnetic field in the examination volume, means for generating switched magnetic field gradients superimposed upon the main magnetic field, means for radiating RF pulses towards the body, control means for controlling the generation of the magnetic field gradients and the RF pulses, means for receiving and sampling magnetic resonance signals, and reconstruction means for forming MR images from the signal samples. According to the invention, the device is arranged to

a) generate a series of MR signals by subjecting at least a portion of the body to an MR imaging sequence of at least one RF pulse and switched magnetic field gradients, the switched magnetic field gradients being selected such that a substantially spherical volume in k-space is sampled along a plurality of radial directions having a non-isotropic angular spacing, the angular density of the radial k-space directions being reduced in the polar regions of the spherical volume, and

b) acquire the MR echo signals for reconstructing an MR image therefrom.

The invention is based on the recognition of the fact, that the scan time can be significantly reduced by thinning out the sampling profiles at the poles of the spherical k-space volume. In situations where the object to be imaged has anisotropic extensions, e.g., extremities, the anisotropic sampling technique according to the invention allows to reduce scan duration with negligible loss in image quality. The amount of reduction depends on the detailed object shape, but can be in the order of at least 10%, but 25% and even more is possible.

Preferably, the switched gradient magnetic fields are selected in accordance with the invention such that the spherical k-space volume is undersampled. A maximum increase in imaging speed is achieved in this way. Because of the anisotropy of the sampling scheme that corresponds to the prolate shape of the imaged object, the undersampling does advantageously not lead to an intolerable level of image artifacts. A good tradeoff between image quality and imaging speed is obtained in accordance with the invention, if the switched gradient magnetic fields are selected such that the density of the radial k-space profiles is reduced in the polar regions of the spherical k-space volume by at least 10%, preferably by at least 25%, as compared to the density in the equatorial regions of the spherical k-space volume.

In a practical embodiment of the invention, the radial k-space profiles determined by the polar k-space coordinates k_{z}, and φ may be selected in accordance with the formulas

Δk_{z}=Δk_{z0}/(1−αsin²(π/2k_{z})) and Δφ=\sqrt{2πΔk_{z}}/\sqrt{1−k_{z}²},

wherein Δk_{z} and Δφ are increments of the polar k-space coordinates, Δk_{z0} is a constant factor determining the overall number of radial profiles, and α is a parameter determining the degree of anisotropy of k-space sampling. The anisotropic arrangement of radial profiles obtained according to these formulas is derived from the known isotropic sampling pattern. The desired reduced sampling density in the polar regions of the spherical k-space volume is regulated by the parameter α. α can range from 0 (isotropic sampling) to almost 1 (massively anisotropic sampling). A larger value of α also means a larger reduction of the overall number of radial profiles: for instance, α=0.5, corresponds to a 25% reduction, whereas α=0.75 corresponds to a 37.5% reduction. By incrementing φ according to the above formula, a homogeneous distribution of profiles is obtained in the azimuthal direction.

The MR imaging sequence applied in accordance with the invention may be an ultrashort echo time (UTE) sequence. An UTE sequence is advantageously employed to observe short-living spin species usually found in cortical bone, tendons, ligaments, menisci, and related tissue. The majority of protons in these tissues exhibits T₂ relaxation times that are too short to be detected by means of conventional imaging sequences. A 3D UTE sequence, which may be applied in accordance with the invention, uses an initial non-selective RF block pulse for excitation. Thereafter, a 3D radial readout magnetic field gradient is switched on to sample the free induction decay (FID). The beginning of the data acquisition coincides with the origin of the spherical k-space volume. Thus, k-space is sampled radially starting at k=0. The endpoints of the radial profiles lie on the surface of a sphere and may be incremented in accordance with the above formulas such that they follow a spiral path with varying turn distance from one pole to the other pole of the sphere. Thereby, the desired anisotropic sampling scheme is obtained. Due to the radial sampling, the center of the spherical k-space volume is heavily oversampled. This makes the technique less susceptible to image artifacts even if undersampling occurs in the peripheral regions of k-space.

The invention not only relates to a device but also to a method for magnetic resonance imaging of at least a portion of a body placed in an examination volume of an MR device. The method comprises the following steps:

a) generating a series of MR signals by subjecting at least a portion of the body to an MR imaging sequence of at least one RF pulse and switched magnetic field gradients, the switched magnetic field gradients being selected such that a substantially spherical volume in k-space is sampled along a plurality of radial directions having a non-isotropic angular spacing, the angular density of the radial k-space directions being reduced in the polar regions of the spherical volume, and

b) acquiring the MR echo signals for reconstructing an MR image therefrom.

A computer program adapted for carrying out the imaging procedure of the invention can advantageously be implemented on any common computer hardware, which is presently in clinical use for the control of magnetic resonance scanners. The computer program can be provided on suitable data carriers, such as CD-ROM or diskette. Alternatively, it can also be downloaded by a user from an Internet server.

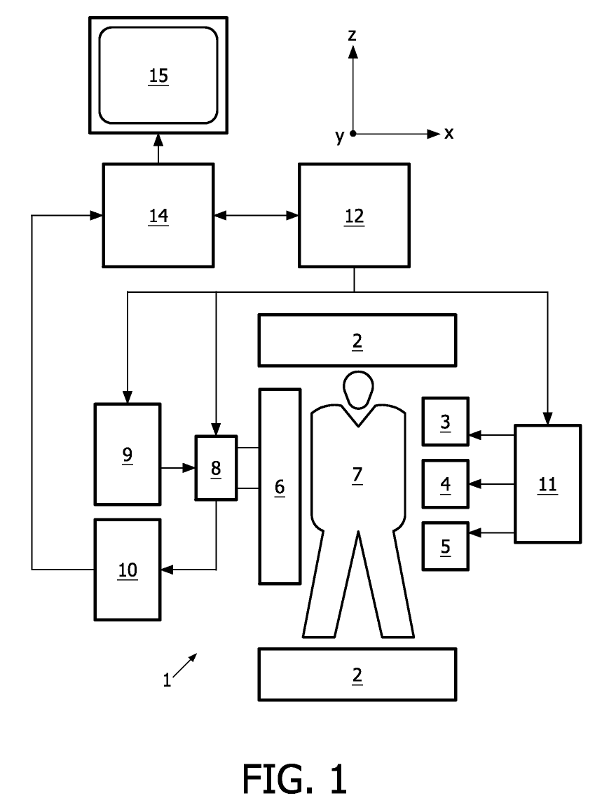

The enclosed drawings disclose preferred embodiments of the present invention. It should be understood, however, that the drawings are designed for the purpose of illustration only and not as a definition of the limits of the invention. In the drawings FIG. 1 shows an MR scanner according to the invention;

FIG. 2 illustrates a conventional 3D radial sampling scheme;

FIG. 3 illustrates a non-isotropc 3D radial sampling scheme according to the invention;

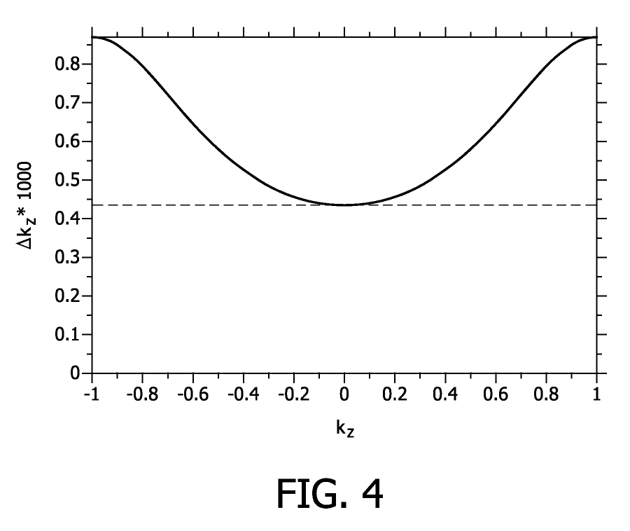

FIG. 4 shows a diagram in which the sampling density is depicted as a function of k_{z}.

In FIG. 1 an MR imaging device 1 in accordance with the present invention is shown as a block diagram. The apparatus 1 comprises a set of main magnetic coils 2 for generating a stationary and homogeneous main magnetic field and three sets of gradient coils 3, 4 and 5 for superimposing additional magnetic fields with controllable strength and having a gradient in a selected direction. Conventionally, the direction of the main magnetic field is labelled the z-direction, the two directions perpendicular thereto the x- and y- directions. The gradient coils 3, 4 and 5 are energized via a power supply 11. The imaging device 1 further comprises an RF transmit antenna 6 for emitting radio frequency (RF) pulses to a body 7. The antenna 6 is coupled to a modulator 9 for generating and modulating the RF pulses. Also provided is a receiver for receiving the MR signals, the receiver can be identical to the transmit antenna 6 or be separate. If the transmit antenna 6 and receiver are physically the same antenna as shown in FIG. 1, a send-receive switch 8 is arranged to separate the received signals from the pulses to be emitted. The received MR signals are input to a demodulator 10. The send-receive switch 8, the modulator 9, and the power supply 11 for the gradient coils 3, 4 and 5 are controlled by a control system 12. Control system 12 controls the phases and amplitudes of the RF signals fed to the antenna 6. The control system 12 is usually a microcomputer with a memory and a program control. The demodulator 10 is coupled to reconstruction means 14, for example a computer, for transformation of the received signals into images that can be made visible, for example, on a visual display unit 15. For the practical implementation of the invention, the MR device 1 comprises a programming for generating an MR imaging sequence with 3D radial sampling of k-space in the above described manner.

FIG. 2 illustrates a conventional 3D radial k-space sampling scheme, wherein k-space is sampled with isotropic angular density. This is obtained by arranging radial profiles on a spiral path over the surface of a sphere. In this case, k_{z} steps are equally spaced, and the azimuthal angle is varied according to Δφ=sin⁻¹(k_{z})\sqrt{Nπ}, with N being the number of radial projections.

In FIG. 3, a 3D radial sampling scheme in accordance with the invention is illustrated. To obtain a reduced angular density in the polar regions of the spherical k-space volume to be acquired, a variable k_{z} increment Δk_{z} is introduced. It is varied according to Δk_{z}=Δk_{z0}/(1−αsin²(π/2k_{z})) . The constant α determines the degree of anisotropic undersampling. It can range from 0 (isotropic sampling) to almost 1 (massively anisotropic sampling). A larger value of α also means a larger reduction of the number of radial profiles: for instance, α=0.5, corresponds to a 25% reduction as it is depicted in FIG. 3, whereas α=0.75 corresponds to 37.5%. For a homogeneous distribution of profiles, azimuthal angle increments have to be varied according to Δφ=\sqrt{2πΔk_{z}}/\sqrt{1−k_{z}²}. A reduction in angular sampling density around the poles of the spherical k-space volume according to the invention, i.e., in k_{z} direction, reduces the imaging volume in the x and y directions. For a given α, the sampling density at the poles is decreased by a factor 1/(1−α), which results in an equatorial FOV reduction of 1/\sqrt{1−α}, e.g., \sqrt{2} for [symbol] =0.5. This illustrates the benefits of the invention in situations where the object to be imaged has anisotropic extensions, e.g., extremities. In such cases, the anisotropic sampling technique according to the invention allows to reduce scan duration with negligible loss in image quality. The amount of reduction depends on the detailed object shape, but can be in the order of 25% and more.

The diagram depicted in FIG. 4 shows the increments Δk_{z} as a function of k_{z} for isotropic sampling (dashed line) and anisotropic radial sampling with [symbol] =0.5 according to the above formula (solid line). As can be seen from the diagram, the increments Δk_{z} are increased in the polar regions (k_{z}<−0.5 or k_{z}>0.5) which means that the sampling density in these regions is reduced correspondingly. It is an important aspect of the invention that the anisotropy of the radial sampling scheme is achieved by a smooth variation of the angular density of the radial k-space profiles from the equatorial region to the polar regions of the spherical k-space volume, as it is illustrated in FIG. 4. An optimal reduction of scan time without intolerable loss of image quality is achieved in this way.

1. A device for MR imaging of a body placed in an examination volume, the device comprising
means for establishing a substantially homogeneous main magnetic field in the examination volume,
means for generating switched magnetic field gradients superimposed upon the main magnetic field,
means for radiating RF pulses towards the body,
control means for controlling the generation of the magnetic field gradients and the RF pulses,
means for receiving and sampling MR signals, and
reconstruction means for forming MR images from the signal samples, the device being arranged to
a) generate a series of MR signals by subjecting at least a portion of the body to an MR imaging sequence of at least one RF pulse and switched magnetic field gradients, the switched magnetic field gradients being selected such that a substantially spherical volume in k-space is sampled along a plurality of radial directions having a non-isotropic angular spacing, the angular density of the radial k-space directions being reduced in the polar regions of the spherical volume, and
b) acquire the MR echo signals for reconstructing an MR image therefrom.

2. The device of claim 1, wherein the device is further arranged to select the switched gradient magnetic fields such that the spherical k-space volume is undersampled.

3. The device of claim 1, wherein the device is further arranged to select the switched gradient magnetic fields such that the density of the radial k-space profiles is reduced in the polar regions of the spherical k-space volume by at least 10%, preferably by at least 25%, as compared to the density in the equatorial regions of the spherical k-space volume.

4. The device of claims 1, wherein the device is further arranged to select the radial k-space profiles determined by the polar k-space coordinates k_{z} and φ in accordance with the formulas
Δk_{z}=Δk_{z0}/(1−αsin²(π/2k_{z})) and
Δφ=\sqrt{2πΔk_{z}}/\sqrt{1−k_{z}²},
wherein Δk_{z} and Δφ are increments of the polar k-space coordinates, Δk_{z0} is a constant factor determining the overall number of radial profiles, and α is a parameter determining the degree of anisotropy of k-space sampling.

5. The device of claims 1-4, wherein the MR imaging sequence is an ultrashort echo time (UTE) sequence.

6. A method for MR imaging of at least a portion of a body placed in an examination volume of an MR device, the method comprising the following steps:
a) generating a series of MR signals by subjecting at least a portion of the body to an MR imaging sequence of at least one RF pulse and switched magnetic field gradients, the switched magnetic field gradients being selected such that a substantially spherical volume in k-space is sampled along a plurality of radial directions having a non-isotropic angular spacing, the angular density of the radial k-space directions being reduced in the polar regions of the spherical volume, and
b) acquiring the MR echo signals for reconstructing an MR image therefrom.

7. The method of claim 6, wherein the switched gradient magnetic fields are selected such that the spherical k-space volume is undersampled.

8. The method of claims, wherein the radial k-space profiles determined by the polar k-space coordinates k_{z} and φ are selected in accordance with the formulas
Δk_{z}=Δk_{z0}/(−αsin²(π/2k_{z})) and
Δφ=\sqrt{2πΔk_{z}}/\sqrt{1−k_{z}²},
wherein Δk_{z} and Δφ are increments of the polar k-space coordinates, Δk_{z0} is a constant factor determining the overall number of radial profiles, and α is a parameter determining the degree of anisotropy of k-space sampling.

9. A computer program for an MR device, the program comprising instructions for generating an MR imaging sequence for sampling a substantially spherical volume in k-space using a plurality of radial k-space profiles having a non-isotropic angular spacing, the angular density of the radial k-space profiles being reduced in the polar regions of the spherical volume.

10. The computer program of claim 9, wherein the program further comprises instructions for selecting the radial k-space profiles determined by the polar k-space coordinates k_{z} and φ in accordance with the formulas
Δk_{z}=Δk_{z0}/(1−αsin²(π/2k_{z})) and
Δφ=\sqrt{2πΔk_{z}}/\sqrt{1−k_{z}²},
wherein Δk_{z} and Δφ are increments of the polar k-space coordinates, Δk_{z0} is a constant factor determining the overall number of radial profiles, and α is a parameter determining the degree of anisotropy of k-space sampling.
